(19) United States
(12) Patent Application Publication
(10) Pub. No.: US 2021/0403347 A1
(43) Pub. Date: Dec. 30, 2021

(54) TREATMENT OF SKIN DISORDERS BASED ON ELECTROLYSED WATER

(71) Applicant: WATERDIAM GROUP LLC, Miami, FL (US)

(72) Inventors: Anthony GINTER, Miami, FL (US); Laurent PUPUNAT, Delemont (FR)

(21) Appl. No.: 17/296,096

(22) PCT Filed: Nov. 21, 2019
(86) PCT No.: PCT/EP2019/082163

§ 371 Date: May 21, 2021

(30) Foreign Application Priority Data
Nov. 21, 2018 (FR) 1871657

(51) Int. Cl. C02F 1/467; A61K 33/14; A61P 17/02

(52) U.S. Cl. CPC C02F 1/4674; C02F 2001/46147; A61P 17/02; A61K 33/14

(57) ABSTRACT

The present invention relates to a method for obtaining electrolysed water comprising the following steps: —Providing running or spring water, optionally comprising sodium chloride (NaCl) at a concentration of 0.5 to 2 g/L —Electrolysing this water by means of an electrolysis module comprising at least one boron-doped diamond electrode attached to a silicon substrate in which the concentration of boron is between 200 ppm (3×10¹⁹ B atoms/cm³) and 1500 ppm (2×10²⁰ B atoms/cm³), the electric current density during the electrolysis process being between 15 and 500 mAh/L water, more preferably between 40 and 250 mAh/L water, and more preferably still between 50 and 200 mAh/L, and the electrolysis period being less than or equal to 60 minutes. The invention also concerns a water obtained according to this method for treating skin disorders and a composition containing such a water and a device for treating skin disorders comprising a vessel containing a water obtained according to the inventive method.

The invention relates to the treatment of the skin disorders such as eczema or atopic dermatitis. The invention relates more particularly to a method for obtaining a water useful for treating the skin disorders comprising a treatment by electrolysis. The invention also concerns the water obtained by this method, its use for the treatment of said skin disorders by direct application or in the form of compositions, and a suitable device for treating said disorders.

The skin disorders, unlike other diseases, have the disadvantage of being visible as well as painful. If we consider a fever blister, this is not very serious because, although it is visible and not very aesthetic, it disappears quickly with time.

It is quite different when we consider inflammatory skin disorders; in addition to being painful, they affect the self-esteem of many patients because the disorder can be spectacularly visible on a large part of the body and is not exactly easy to look at. This is the case with eczema, which is defined as an inflammation of the skin. Eczemas, as there are of several types, can be allergic or contact or nummular in nature, presenting as isolated patches of the irritated skin located most often on the arms, the buttocks, the back or the lower legs.

Atopic dermatitis is an itchy and inflammatory dermatitis that can be accompanied by asthma and can spread over the whole body in large patches.

Another disadvantage of these diseases, apart from the pain, is that they are chronic and often appear and disappear depending on the psychological or general state of the patient.

All of these characteristics mean that a single short-term treatment is generally not possible to treat these diseases. Indeed, as these diseases are chronic, they require regular treatment.

Several solutions have been developed and as these diseases affect the skin, the topical route is the most logical.

Without being exhaustive, mention may be made of the application WO200710766. The object of the invention is the use of the E-3-[3-[N-(4-methoxybenzenesulfonyl)-N-isopropylamino]phenyl]-3-(3-pyridyl)propenohydroxamic acid of formula (I); or one of its pharmaceutically acceptable salts, pharmaceutically acceptable solvents or hydrates, for the preparation of a drug intended to prevent and/or treat the cutaneous inflammatory pathologies, as well as the pharmaceutical compositions, in particular dermatological, comprising the E-3-[3-[N-(4-methoxybenzenesulfonyl)-N-isopropylamino]phenyl-3-(3-pyridyl) propenohydroxamic acid, or one of its pharmaceutically acceptable salts, pharmaceutically acceptable solvents or hydrates, for the treatment and/or the prevention of cutaneous inflammatory pathologies.

Or the application WO2014049300, the object of the invention of which is a topical pharmaceutical composition, in particular dermatological, comprising laropiprant, an ester or a pharmaceutically acceptable salt thereof, and its use for treating dermatological disorders, in particular the rosacea.

But also the application WO2018041751, which relates to a sulphated polysaccharide extracted from a red alga of the species Haliptilon subulatum, or a salt thereof as an active ingredient. More particularly, it relates to a sulphated polysaccharide extracted from a red alga of the species Haliptilon subulatum, or a salt thereof as an active ingredient, for its use in the modulation of the immune response in humans or animals, in particular in the prevention and/or the treatment of inflammatory diseases, in particular in the skin.

Although effective, these solutions have two major disadvantages due to the chronicity of this type of disorder: the regular use of chemical products in the form of creams or compositions can be irritant in the long term, and regular use can lead to resistance or addiction, like the cortisone-based treatments for eczemas.

Thus there is a real need for alternative methods or compositions with a non-irritant profile, allowing the least possible use of products that can generate resistance or addiction and allowing to treat these inflammatory skin diseases that are often incapacitating or stressful for the health of the patients.

One of the aims of the invention is therefore to remedy the aforementioned disadvantages and to meet the aforementioned needs by providing an electrolysed water for use in the treatment of inflammatory skin diseases.

More particularly, the envisaged use of the electrolysed water according to the invention is in the treatment of eczema or atopic dermatitis.

The electrolysed water according to the invention is obtained by implementing a three-step method which starts with the use of a running water or spring water optionally supplemented with sodium chloride (NaCl) at a concentration between 0.5 to 2 g/L followed by an electrolysis of said water by means of an electrolysis module comprising at least one boron-doped diamond electrode attached on a silicon substrate in which the boron concentration is between 200 ppm (3×10¹⁹ B atoms/cm³) and 2000 ppm (3, 52×10²⁰ B atoms/cm³), in particular between 200 ppm (3×10¹⁹ B atoms/cm³) and 1500 ppm (2×10²⁰ B atoms/cm³), said module subjecting the water to an amount of current (current density) during the electrolysis process between 15 and 500 mAh/L of water, more preferably 40 to 250 mAh/L of water, even more preferably 50 to 200 mAh/L, the duration of the electrolysis being less than or equal to 60 minutes and in particular between 15 and 60 minutes, and more particularly between 15 and 30 minutes.

The electrolysis can be carried out at once continuously or cyclically, during which the number of cycles is between 2 and 12 times per 24-hour period and spaced at least half an hour apart.

The invention also relates to an electrolysed water obtained by the method described above.

The invention also relates to a composition comprising an electrolysed water according to the invention for use as a drug in the treatment of inflammatory skin diseases.

Preferably, the treatment of inflammatory diseases by this composition relates to eczema or atopic dermatitis.

The composition comprising the electrolysed water according to the invention, mainly composed and all or part of water, it consists of 95 to 100% of electrolysed water and 0 to 5% of an excipient and/or emulsifier.

The composition according to the invention may be in the form of a cream, a gel, a dressing, a patch.

The composition according to the invention for treating the skin disorders may consist of a mask which can be applied to the skin or the face, said mask being obtained from a powder such as a clay, an exfoliant, a powdered plant extract or a clay containing activated carbon and to which electrolysed water obtained according to the method described above has been added.

Furthermore, when the composition according to the invention is exclusively composed of electrolysed water, the latter can be added to a device containing it. Said device being a swimming pool, a spa, a bathtub or a shower.

The electrolysed water according to the invention for use in the treatment of the inflammatory skin diseases is used over a period of 5 to 60 minutes of exposure.

The electrolysed water is obtained by means of an electrolysis module enabling the above-described method to be carried out, said module being optionally present in a fixed or mobile manner in a device such as a swimming pool, a spa, a bathtub or a shower.

It may also be present in a conventional water distribution system, preferably at the outlet of a distribution system such as a tap.

The attached drawings illustrate the invention:

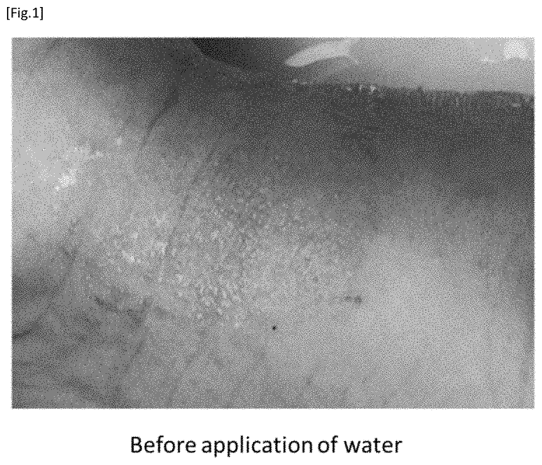

FIG. 1 represents a portion of the ankle of a child suffering from eczema before application of the water according to the invention;

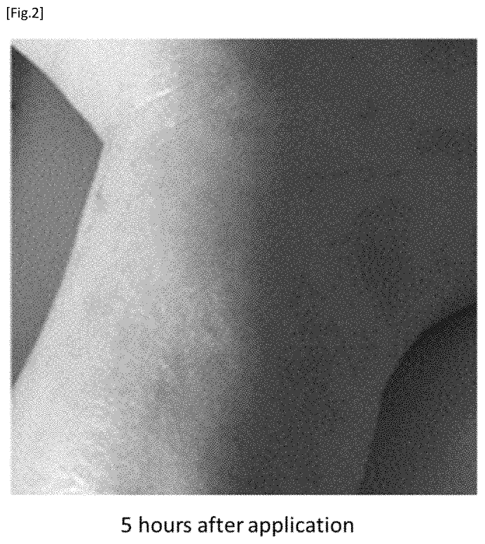

FIG. 2 represents the portion of the ankle of FIG. 1 five hours after application of the water according to the invention;

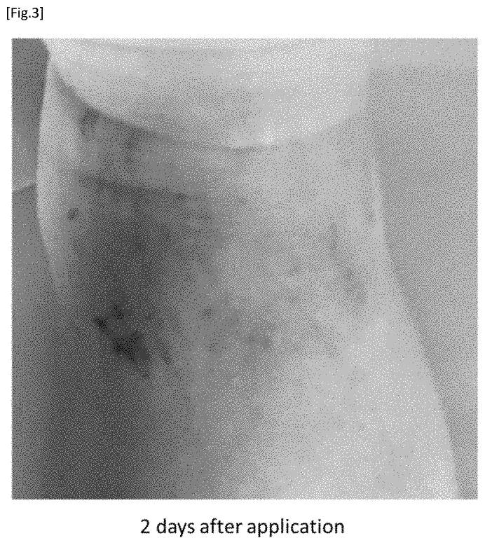

FIG. 3 represents the portion of the ankle of FIG. 1 two days after application of the water according to the invention;

FIGS. 1 to 3 represent the effectiveness of the electrolysed water according to the invention in the treatment of eczema on the skin of the feet of a child before treatment and after treatment by bathing in the electrolysed water.

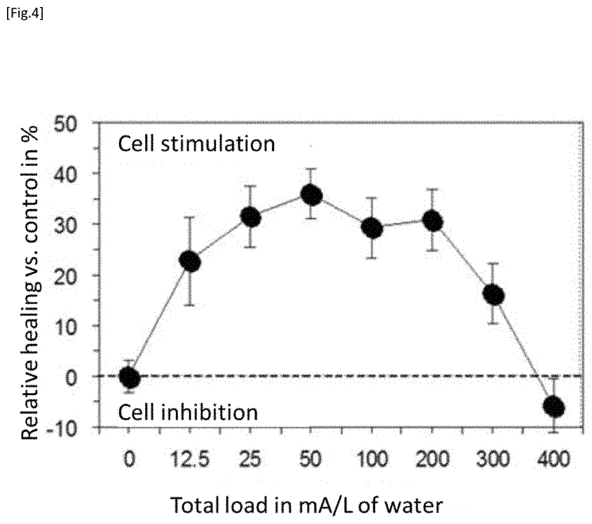

FIG. 4 represents the action of a composition according to the invention comprising an electrolysed water obtained by electrolysis of water by boron-doped diamond electrodes (Electrodes A, 1200 ppm boron) on a silicon substrate on the healing process of a wound.

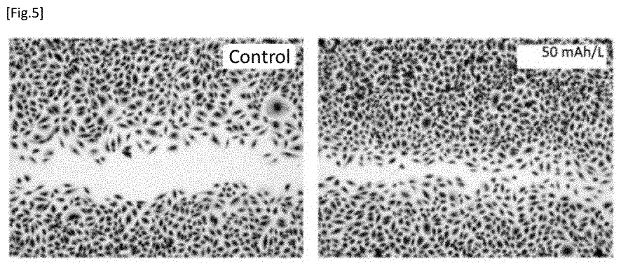

FIG. 5 represents a comparative test between a treatment with a conventional water (control) and an electrolysed water in a composition according to the invention on the healing process.

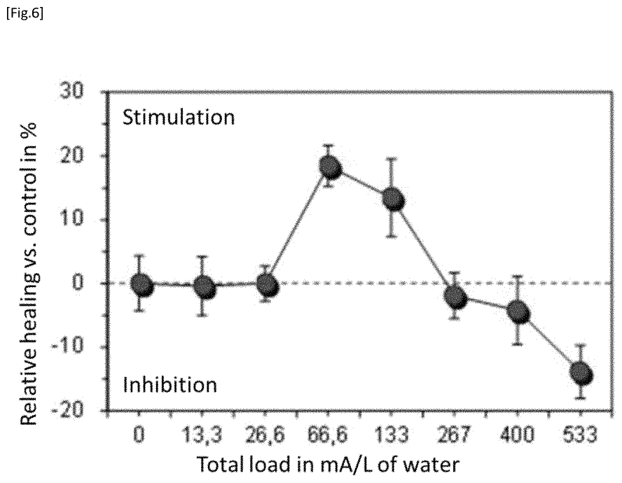

FIG. 6 represents the action of an electrolysed water obtained by electrolysis of water with boron-doped diamond electrodes (Electrodes B, 2500 ppm boron) on a silicon substrate on the healing process of a wound.

Eczema is an itchy dermatitis that manifests itself as a non-contagious inflammation of the skin with redness, fine blisters, scales and itching. The people who are affected experience periods when the disease is manifested by flare-ups, during which the symptoms worsen and then subside. These episodes of flare-ups and lulls or remissions are spread over a more or less long period, especially in the adolescent, which can be very disturbing for them.

Moreover, the aesthetics of eczema, depending on its severity and the nature of the resulting inflammation, can range from bright red patches spread over a large or small area of the body, to purulent and irritating pimples on the face or arms or legs.

In addition to the unsightly appearance of these manifestations and the resulting discomfort, there is also pain.

Among all the skin diseases, eczema is the most common: it accounts for up to 30% of dermatology consultations, affecting 15% to 30% of children and 2% to 10% of adults. The reported cases of eczema have doubled or even tripled in the last 30 years. There are several types of eczema which can be divided into two categories:

the contact or allergic dermatitis, which are characterised by eczema lesions after a contact of the skin with certain substances called allergens, which may be chemicals, clothing or sometimes metals. Eczema may appear within minutes or 3 to 10 days depending on the degree and the time of exposure to the allergen. The allergic reaction may appear very early in the individual's life or much later,

atopic dermatitis characterised by allergic reactions mediated by antibodies called IgE in contact with allergens that are normally harmless to the rest of the population (dust, pollen, animal hair, etc.). The people with atopy often have a variety of allergic reactions, such as hay fever, hives, asthma or food allergies, either simultaneously or alternately.

In the infant, the lesions appear on the cheeks, the forehead and the scalp and extend to the extension faces of the arms and legs and the trunk. They are characterised by dry and rough or oozing and crusty redness, which has the disadvantage of being itchy and irritating, as atopic dermatitis lesions often predominate in the flexion creases of the elbows and the knees or even the wrists.

In the adolescent and the adult, the lesions are mainly located on the face, neck and limbs. They are often thickened and sometimes purulent.

Corticosteroid creams or ointments, mainly cortisone, are applied to the areas to be treated, which reduces itching and inflammation.

The creams and ointments that have a strong steroid effect are used to relieve the severe irritation, but only for a short period of time, as in the long term they lose their effectiveness and can thin the skin.

The antihistamines are also used for itching, but they have a limited duration of action and require several doses.

The immunomodulating drugs are also an alternative to corticosteroids and have the effect of reducing the activity of the immune system (and therefore inflammation) with varying degrees of side effects. It should be noted drugs as the Tacrolimus or the Pimecrolimus. In the case of more severe disorders, the Cyclosporine can be considered. The Cyclosporine is a fungal agent with immunomodulatory properties, it is not without significant side effects on the liver or the blood pressure and can only be used for short periods.

Thus, it can be seen that these inflammatory skin diseases are not without impact on the state of health of the patients and that the therapeutic arsenal available to treat these diseases is not without generating constraints in the long term.

The present invention concerning a particular electrolysed water obtained by a method involving particular boron-doped diamond electrodes attached on silicon enables to solve the problems or disadvantages mentioned above. It has the following advantages and improvements:
It does not rely on conventional drugs which, when used for a long period of time, cause an addiction or a resistance due to the nature of the disease (its recurrence),
It is minimally or non-invasive,
The source of the products used is almost unlimited in some cases and easily accessible,
It does not require a prescription or special precautions when taken or used,
The portability and ease of obtaining the composition according to the invention is also an advantage,
Its production cost is quite low,
The possibility of “reactivating” the composition into an active or therapeutic state easily limits its expiry date considerably,
The absence of allergies or addiction to the products of the composition.

The electrolysis module for carrying out the method according to the invention in order to prepare the electrolysed water useful for the treatment of inflammatory skin diseases comprises at least one, preferably at least two boron-doped diamond electrodes which are attached to a silicon substrate.

The active or contact area of each electrode is between 10 and 100 cm², preferably 60 to 80, more preferably about 70 cm².

The boron doping of the diamond electrode also has an effect on the properties of the water obtained; the boron concentration is in particular between 200 ppm (3×10¹⁹ B atoms/cm³) and 1500 ppm (2×10²⁰ B atoms/cm³).

This concentration of boron, together with the nature of the diamond electrodes to silicon, gives it properties that allow it to operate at a potential of between −1V and −2V on the cathodic polarisation and at +2V and +4V on the anodic polarisation, compared with a reference electrode of platinum.

Without being bound by theory, the fact of accessing much higher operating overvoltages than the classic and expensive platinum electrodes, enables to obtain a water having an interesting therapeutic potential for the skin diseases. The physico-chemical interaction between the electrodes useful for the invention and the electrolysed water molecules allows a functionalization of the water which is not easily characterized with the current techniques but whose therapeutic effects on the skin are directly observable, as shown in the attached figures. The water produced by the method of the invention has therapeutic and biological properties and shows an undeniable activity in comparison with a conventional water not electrolysed according to the inventive method.

The electrolysis module is connected to a power supply module and is open to a flow of water that will pass through it. In order to operate correctly and not to compromise the correct working conditions, the electrodes are supplied with direct current by the power supply module which is connected to the electrolysis module providing a direct current to said electrode, generally this is set between 1.5 A and 3 A. If polarity reversal is required, this can be done automatically by the power supply module.

The water can be supplied from a variety of sources but must pass through the electrolysis module without the module being able to operate without water. The electrolysis module may be permanently traversed by the water depending on the device in which it is located. There is an internal measurement system or sensor of hydraulic flow that interacts with the power module and allows the electrolysis module to be switched on, off or on standby in the absence or presence of water and thus to activate the electrolysis.

Advantageously, the water electrolysis module according to the present invention can be operated in automatic mode or can be activated or deactivated on demand, manually or by means of a remote control system.

A further advantage of the present invention is that the water electrolysis module does not necessarily need to be permanently activated, but can usefully be activated periodically, i.e. at convenient intervals, preferably but not necessarily at regular intervals. It has been found that the electrolysis of the water at regular intervals allows the water to remain therapeutically active for a long period.

Activating the electrolysis module to subject the water to an electrolysis in a cyclic manner and in which the number of cycles is between 2 to 12 times over a 24 hour period and spaced at least half an hour apart results in a water with a therapeutic potential. The electrolysis module allows the implementation of the method for preparing the electrolysed water according to the invention, characterised by the implementation of the following steps.

In a first step, a running or spring water is provided, to which a conductivity salt such as the sodium chloride (NaCl) is optionally added at a concentration of 0.5 to 2 g/L. In a second step, this water is subjected to an electrolysis by a device comprising at least one boron-doped diamond electrode attached on a silicon substrate in which the boron concentration is between 200 ppm (3×10¹⁹ B atoms/cm³) and 1500 ppm (2×10²⁰ B atoms/cm³), said module subjecting the water to an amount of current during the electrolysis process of between 15 and 500 mAh/L of water, more preferably 40 to 250 mAh/L of water, even more preferably 50 to 200 mAh/L, the duration of electrolysis being between 15 and 60 minutes.

As mentioned above, this electrolysis can be carried out cyclically, with the number of cycles between 2 and 12 times over a 24-hour period and spaced at least half an hour apart. In the case of a relatively small quantity of water used, it can be electrolysed only once for the time mentioned and between 15 and 30 minutes in the case of a single or unique use.

An object of said invention is therefore water as a product obtained by the obtaining method. The water thus obtained by the electrolysis method of the invention is suitable for use in the treatment of inflammatory skin diseases and more particularly for use in the treatment of eczema or atopic dermatitis.

The resulting water may be used in different forms or compositions or within different devices.

The devices used may be selected from a swimming pool, a spa, a bathtub or a water distribution system such as a tap or a shower or a suitable ambulatory treatment device. The electrolysis module used will then be integrated into an existing water circuit in order to prepare the electrolysed water, for example in the swimming pools or the spas, or fitted and connected as a mobile device to a hydraulic circuit in connection with a tap if a shower or a bathtub is considered. In the case of an ambulatory treatment device, an electrolysis device and a water tank of a certain volume can be connected or assembled in a closed circuit with an applicator body or tap, a recovery device can also be associated so as to prevent the treatment electrolysed water from being washed down the drain and its recycling to the tank for a further electrolysis.

The nomadic or mobile or even fixed mode of use and its adaptability to existing devices to produce the electrolysed water useful in the treatment of the inflammatory skin diseases is an advantage of the present invention.

The initial water subjected to the electrolysis, whatever its source, is purified and potentiated by the electrolysis. It is therefore not necessary to use a distilled water, as the electrolysis method of the invention provides a natural anti-bacterial and fungicidal effect, enabling to obtain a quality water.

Any water, whether natural water, spring water or municipal water, can be used in the method for obtaining the electrolysed water according to the invention. The advantage and the flexibility of use of the electrolysis module according to the invention enables to obtain from a common water source a less expensive treatment because it does not require conventional chemical products in the treatment of the inflammatory skin diseases, while avoiding the side effects of said chemical products.

In the case of a use in devices such as swimming pool, spa, bathtub etc., the advantage is that a large surface area can be treated; it can involve the whole body or all or part of the body, especially effective when the inflammatory disease is localised in different areas; this avoids the use of large quantities of cream.

The treatment can be carried out in a bath for a duration of exposure ranging from 5 to 45 minutes and repeated more than once a day until the inflammatory symptoms disappear or are reduced.

In the case of the treatment of a child suffering from severe eczema on the upper and lower limbs and the back as shown in FIGS. 1 to 3, the treatment is most effective after two days and as early as 5 hours after exposure for fifteen minutes to a bath comprising the electrolysed water according to the method of the invention. Before getting into the bathtub containing the water, a very high density of inflammation of the skin is observed in the foot, which diminishes 5 hours after exposure to the bath, then two days after the bath the inflammatory part seems to be in the form of a crust which is still reddish but no longer causes itching, which is conducive to the formation of crusts which will then fade.

The electrolysed water obtained by the method according to the invention can also be present in more conventional compositions which can be found on sale in the pharmacies or parapharmacies or even the supermarkets, said composition comprising 95 to 100% of electrolysed water and 0 to 5% of an excipient and/or emulsifier.

Said composition being for use as a drug in the treatment of inflammatory skin diseases, more particularly eczema or atopic dermatitis.

It may be in the form of a cream, a gel, a dressing or a patch.

In the composition according to the invention, the excipients and/or natural or synthetic emulsifiers selected from petrolatum, glycerine, paraffin, cetearyl glucose, beeswax or rice wax, soya lecithin, sugar esters, glyceryl stearate, olive oil derivatives or a mixture thereof.

Another usable galenic form of the composition according to the invention for treating the skin disorders may consist of a mask which can be applied to the skin or the face, said mask being obtained from a powder such as a clay, an exfoliant, a powdered plant extract or a clay containing activated charcoal and added with electrolysed water obtained according to the method described above. The person skilled in the art will know how to adapt the ratio between powder and electrolysed water according to the invention in order to obtain the desired consistency of the mask.

Depending on the type of cream to be obtained and its desired penetrating power, it can be added to the cream a higher or lower percentage of electrolysed water than the emulsifiers, or vice versa. In the case of a water-in-oil emulsion, also known as W/O, the quantity of oil is greater than the quantity of water. The emulsion thus obtained is very nourishing, moisturising and protective because it creates a lipidic film on the skin. It is ideally used for the dry skins or the night creams.

In the case of an oil-in-water emulsion, also known as O/W, the quantity of water is greater than the quantity of oil. This type of emulsion is nourishing and moisturising. It is ideally used to make day creams, body milks, it can also be integrated into patches or bandage-type adhesives.

Another potential galenic form for the composition according to the invention can be an aqueous gel also called hydrogel. A hydrogel is a gel in which the swelling agent is the water. The matrix of a hydrogel is generally a network of polymers which are insoluble in the water, but are enable of substantial swelling in the presence of a large amount of water or aqueous solutions.

Thus several formulations or shaping of the composition according to the invention may be possible depending on the needs of the patient to be treated and the extent of the inflammation process. It may be in the form of cream, gel or emulsion or even in the form of a bandage-type adhesive or a patch for the skin.

Depending on the type of emulsion sought or the composition sought, the composition according to the invention in the case of a so-called O/W composition (oil in water) comprises from 95 to 100% of electrolysed water and from 0 to 5% of an excipient and/or emulsifiers.

It is not excluded in the present invention that electrolysed water is not the majority compound; this is the case of a so-called W/O (water in oil) composition, said composition comprises from 70 to 90% of an excipient and/or emulsifier and from 10 to 30% by weight of electrolysed water.

Another potential galenic form for the composition according to the invention may be an aqueous gel also called hydrogel. A hydrogel is a gel in which the swelling agent is the water. The matrix of a hydrogel is generally a network of polymers which are insoluble in the water, but are capable of substantial swelling in the presence of a large amount of water or aqueous solutions.

The examples of images and devices according to the invention presented, as well as the various embodiments mentioned, in no way limit the scope of the invention as claimed, they are given by way of example in order to better understand the invention.

Finally, it is clear that the embodiments are only particular illustrations and are in no way limiting the fields of application of the invention.

### EXAMPLES

### Example 1: Study of the Effects of the Boron Concentration of the Electrode on the Efficiency of the Healing of the Wound

The rate of healing of fibroblasts as a function of the boron content of the electrodes used to electrolyse the culture water was studied.

The boron-doped electrodes (hereafter referred to as BDD/Si electrodes) implemented in this experiment have the following characteristics:

BDD/Si electrodes: boron-doped diamond film on silicon substrate:
Substrate: single crystal silicon (100), resistivity 100 mohm·cm
BDD film: polycrystalline, thickness ˜2-3 μm, doping 1200 ppm boron (Electrode A) or 2500 ppm boron (Electrode B),

The electrodes A and B were manufactured using the same HF-CVD (Hot Filament Chemical Vapor Deposition) diamond film growth protocol. They are identical in every respect and differ only in their respective boron content.

a) The Protocol for Implementing the Electrodes on the Culture Water is as Follows:

A 2.5 L tank contains 15° C. city water. This water is pumped at a fixed rate of 200 L/h through an electrolysis module and then returned to the tank in a closed circuit. The electrolysis module uses 2 electrodes spaced 1 mm apart and with an active surface area of 70 cm². The electrolysis current is 2 A for working periods t=0-1-2-5-10-20-30-40 min so as to achieve electrolysis loads of 0 to 533 mAh/L. The tank water is kept at a constant temperature of 20° C. during the test.

The electrolysed water is sampled at the outlet of the electrolysis module and then immediately sterile filtered (0.2 μm porous membrane filters) and added to the culture medium of the fibroblasts at a 1:4 dilution (=25% concentration).

b) The Culture Protocol is as Follows:

Fibroblasts: L-929 (mouse fibroblasts; ACC 173; DSMZ); internal passage P52-53; recommended according to EN ISO 10993-5: 2009). The cells are incubated and mass cultured in a 37° C. incubator with a controlled closed environment containing 5% of CO2 and 95% air. The culture medium is the RPMI 1640 with 10% of physiological bovine serum, 100 Unit/mL of penicillin and 100 μg/mL of streptomycin.

c) The Protocol for Studying the Regeneration/Healing of the Cells is as Follows:

Use of silicone culture inserts (ibidi GmbH, München). When this insert is placed in a culture medium, it forms 2 culture tanks separated by a 500 μm thick wall. The cells are grown in both tanks and the silicone insert is removed. This results in two perfectly defined culture patches and spaced apart of precisely 500 μm.

For the experiments, L-929 type cells were obtained from 80 to 90% by mass of suspension cultures at a density of 500,000 cells/ml. 100 μL of suspension are introduced into each culture insert tank. The cells are grown for 24 hours to obtain homogeneous populations in each of the two tanks of each insert. Then, the insert is gently removed, leaving a 500 μm free space separating the two culture media.

The electrolysed water is injected up to 500 μL to 1,500 μL of fresh culture medium (1:4 dilution). The culture media were grown fresh for 24 hours. Then, layers of cells were attached by a treatment with methanol for 2 min and stained by means of a Coomassie-Giemsa solution according to Romanowsky.

The separation space was photographed via a 27″ screen in order to observe at 5 points along the space, the speed of approach of the two media, up to the junction (FIG. 5).

d) Results

The results with the electrolysed water with the BDD electrode A (1200 ppm B boron doping) show a significant acceleration of the healing rate compared to the sterile water without electrolysis, up to more than 30% from 25 mAh/L to 200 mAh/L and then a decrease (FIG. 4).

With the BDD electrode B (approx. 2500 ppm B), an acceleration of the healing is observed, but much lower than with the electrodes A (FIG. 6).

### Example 2: Boron Content of the Electrolysed Water

The protocol for electrolysis of the tap water is as follows:

The water used is a city water.

The electrolysis protocol is as follows:

The city water is pumped at a flow rate of 90 L/h through an electrolysis module equipped with 2 BDD electrodes A as defined in example 1 (1200 ppm B) spaced 1 mm apart, having 12.5 cm² of active surface.

The applied current is 2.4 A. The water sample is collected directly from the outlet of the electrolysis module (open loop, so-called lost water operation).

The results are reported in the tables below:

Sample 1: raw city water

Samples 2 and 3: electrolysed water with electrodes A (1200 ppm boron)

| Sample | Boron | Si |
|---|---|---|
| 1 raw city | 31.5 | 4.7 |
| 2 low flow mini cell 2.4A | 34 | 4.54 |
| 3 high flow mini cell 2.2A | 34.2 | 4.54 |
| | μg/L | mg/L |
| release | 2.60 | μg B/L |
| medium flow | 90 | L/h |
| medium current | 2.3 | A |
| | 101.74 | μg B/Ah |

The electrolysis of the water with the electrodes A causes a small but noticeable increase in the boron concentration in the electrolysed water compared to the raw water: approximately +101.74 microgram boron/Ah of applied electrical charge. The boron measurements in the water are performed by Inductively Coupled Plasma Mass Spectrometry (ICP-MS).

1-13. (canceled)

14. A method for obtaining an electrolyzed water comprising:
providing a running or spring water, optionally comprising sodium chloride (NaCl) at a concentration between 0.5 and 2 g/L, and
electrolyzing said water by means of an electrolysis module comprising at least one boron-doped diamond electrode attached to a silicon substrate in which the concentration of boron is between 200 ppm (3×10¹⁹ B atoms/cm³) and 2000 ppm (3.52×10²⁰ B atoms/cm³), in particular between 200 ppm (3×10¹⁹ B atoms/cm3) and 1500 ppm (2×10²⁰ B atoms/cm³).

15. The method according to claim 14, wherein the electrolysis is carried out cyclically, a number of cycles being between 2 and 12 cycles per 24-hour period, each electrolysis cycle being spaced from a preceding or following cycle by at least 30 minutes.

16. Water obtained by:
providing a running or spring water, optionally comprising sodium chloride (NaCl) at a concentration between 0.5 and 2 g/L, and,
electrolyzing said water by means of an electrolysis module comprising at least one boron-doped diamond electrode attached to a silicon substrate in which the concentration of boron is between 200 ppm (3×10¹⁹ B atoms/cm³) and 2000 ppm (3.52×10²⁰ B atoms/cm³), in particular between 200 ppm (3×10¹⁹ B atoms/cm′) and 1500 ppm (2×10²⁰ B atoms/cm³).

17. A method of treatment of inflammatory skin diseases, in particular the treatment of eczema or atopic dermatitis, comprising administering to a subject in need, water obtained by:
providing a running or spring water, optionally comprising sodium chloride (NaCl) at a concentration between 0.5 and 2 g/L, and
electrolyzing said water by means of an electrolysis module comprising at least one boron-doped diamond electrode attached to a silicon substrate in which the concentration of boron is between 200 ppm (3×10¹⁹ B atoms/cm³) and 2000 ppm (3.52×10²⁰ B atoms/cm³), in particular between 200 ppm (3×10¹⁹ B atoms/cm³) and 1500 ppm (2×10²⁰ B atoms/cm³),
is administered to a subject in need thereof.

18. A method of treatment of inflammatory skin diseases, especially eczema or atopic dermatitis, wherein a composition comprising administering to a subject in need, water obtained by:
providing a running or spring water, optionally comprising sodium chloride (NaCl) at a concentration between 0.5 and 2 g/L, and,
electrolyzing said water by means of an electrolysis module comprising at least one boron-doped diamond electrode attached to a silicon substrate in which the concentration of boron is between 200 ppm (3×10¹⁹ B atoms/cm³) and 2000 ppm (3.52×10²⁰ B atoms/cm³), in particular between 200 ppm (3×10¹⁹ B atoms/cm³) and 1500 ppm (2×10²⁰ B atoms/cm³),
is administered to a subject in need thereof.

19. The method according to claim 18, wherein the composition comprises 95 to 100% of electrolyzed water and 0 to 5% of an excipient and/or emulsifier.

20. The method according to claim 19, wherein the composition is packaged for application in the form of a cream, a gel, a dressing or a patch.

21. An inflammatory skin disease treatment device comprising a tank containing a water that has been obtained by:
providing a running or spring water, optionally comprising sodium chloride (NaCl) at a concentration between 0.5 and 2 g/L, and
electrolyzing said water by means of an electrolysis module comprising at least one boron-doped diamond electrode attached to a silicon substrate in which the concentration of boron is between 200 ppm (3×10¹⁹ B atoms/cm³) and 2000 ppm (3.52×10²⁰ B atoms/cm³), in particular between 200 ppm (3×10¹⁹ B atoms/cm³) and 1500 ppm (2×10²⁰ B atoms/cm³).

22. The device according to claim 21, wherein an electrolysis module performs the electrolyzing directly in the tank.

23. The device according to claim 22, wherein the electrolysis module is removable from the tank.

24. The device according to claim 21, wherein the device further comprises means for circulating the water in the tank.

25. The device according to claim 21, wherein the device further comprises means for applying the water contained in the tank to a skin area of a patient by soaking said skin area in a bath.

26. The device according to one of claim 21, wherein the device further comprises means for applying the water contained in the tank to a skin area of a patient by spraying or flowing at a controlled rate onto said skin area, the device further comprising means for recovering and recycling the water sprayed or flowed towards the tank.
